(19) United States
(12) Patent Application Publication
(10) Pub. No.: US 2026/0250229 A1
(43) Pub. Date: Aug. 27, 2026

(54) PROCESS FOR THE CONTINOUS UPGRADING OF HEAVY BY-PRODUCTS FROM ACRYLIC ACID PRODUCTION

(71) Applicant: Arkema France, Colombes (FR)

(72) Inventors: Frederic SANDRE, Saint Avold Cedex (FR); Marc ESCH, Saint Avold Cedex (FR); Fanny DANTON, Saint Avold Cedex (FR)

(21) Appl. No.: 18/878,115

(22) PCT Filed: Jun. 20, 2023
(86) PCT No.: PCT/FR2023/050913

§ 371 Date: Dec. 23, 2024

(30) Foreign Application Priority Data
Jun. 24, 2022 (FR) FR2206330

(51) Int. Cl. C07C 51/42

(52) U.S. Cl. CPC C07C 51/42

(57) ABSTRACT

A process for regenerating acrylic acid (AA), by thermal cracking, from heavy by-products (residues referred to as AAHP) from an AA production unit, with a view to recycling them in the acrylic acid production plant. This process includes two steps operating continuously while improving the current performance of cracking plants.

### FIELD OF THE INVENTION

The present invention relates to a process for regenerating acrylic acid (AA), by thermal cracking, from heavy by-products (residues referred to as AAHP) from an AA production unit, with a view to recycling them in the acrylic acid production plant. This process consists of two steps operating continuously while improving the current performance of cracking plants.

### TECHNICAL BACKGROUND

Under the effect of the temperature during the distillation steps, the manufacture of acrylic acid is accompanied by the formation of heavy compounds, derivatives of the addition of compounds having a nucleophilic property to the double bond of the unsaturated carbonyl-containing monomers, by the Michael reaction. Compounds having a boiling point higher than that of the acrylic monomer manufactured are referred to as “heavy” compounds.

In the case of an AA production unit, these are essentially:
derivatives of the addition of acrylic acid to the double bond of another acrylic acid molecule: 3-acryloyloxypropionic acid, also called “acrylic acid dimer” or “AA dimer”;
derivatives of the addition of acrylic acid to the double bond on an AA dimer molecule, to form the “AA trimer”, and other oligomers formed by successive additions of acrylic acid to the double bonds of the preceding AA oligomers; and
derivatives of the addition of carboxylic acids, formed as by-products of the acrylic acid, or of water to the double bond of the AA or of the abovementioned oligomers.

The recovery of upgradable monomers from heavy Michael derivative compounds is difficult in the case of heavy products originating from an AA production unit. Specifically, during the thermal cracking process that regenerates acrylic acid, which is distilled and upgraded, a residue remains, the viscosity of which increases greatly when high cracking efficiencies are sought, until it can no longer be extracted from the cracking reactor.

The main factor limiting the efficiency of the regeneration of the compounds derived from the Michael reaction contained in the heavy streams from AA plants is the increase in the viscosity of the heavy residue obtained at the bottom of the cracker, when the fraction rich in acrylic monomers has been evaporated.

The evaporation of light compounds during the cracking results in a concentration of the heavy products in the residue stream and an increase in the viscosity of this stream. However, the residue has to remain sufficiently fluid after cooling to be transported and then treated for the purpose of destroying it.

In the case where there is production of light esters (methyl acrylate (MA) or ethyl acrylate (EA)) close to the AA production unit, a co-cracking of the respective heavy products can improve the situation, making the cracking residue more fluid. The proposed solution makes it possible to recover the maximum amount of AA per cracking operation while managing the viscosity of the residue formed without being dependent on another production unit.

Thus, in document EP 717 031, it has been shown that it is possible to improve the efficiency of the recovery of these upgradable noble products, if the cracking is carried out with a mixture of heavy products originating from an AA production unit and from an acrylic ester (EA) production unit, compared to the individual cracking of the heavy streams from these units. The effect of the addition of heavy products originating from the ester units (EAHP) to the heavy products originating from an AA unit (AAHP) is to reduce the viscosity of the final residue. The cracking reaction is carried out with mixtures having an AA heavy products/ester heavy products ratio of 9/1 to 1/9, at a temperature of 180° C. to 220° C., under atmospheric pressure, for a residence time of 0.5 to 3 hours. In this process, the cracking and the evaporation of the light compounds generated are carried out in a reactor, then the gas stream generated is sent into a distillation column and, finally, the bottom stream from the distillation column is recycled to the reactor. On the other hand, as the light fraction obtained by cracking consists mainly of AA and ester acrylic monomers, which are particularly sensitive to polymerization, the distillation stage necessarily has to be carried out under reduced pressure, so as to reduce the temperature, in order to prevent the formation of polymer in the column. Furthermore, the rectifying plates of the distillation column bring about the efficient separation of the polymerization inhibitors entrained in the gas mixture, . . . which flow back to the column bottom, and consequently it is necessary to introduce fresh polymerization inhibitors at the column top, in order to prevent the formation of polymers in the upper part of the column.

For this reason, the reaction stage, carried out under higher pressure, and the distillation stage, carried out under reduced pressure, have to be separated. The installation for carrying out the process thus has to be equipped with a reactor and with a top condenser, which are operated at the same pressure, and with a distillation column operated at reduced pressure, fed with the condensed product, and comprising a boiler at the bottom and, at the top, a condenser, an item of reflux equipment and a feed of inhibitors. This arrangement is complicated and expensive.

Moreover, the co-cracking of AA heavy products mixed with EA heavy products leads to operating constraints. Specifically, when the ester unit is in shutdown, the cracking operation must be shut down. This leads to economic losses.

In other scenarios, the AA heavy products are thermally cracked batchwise without the addition of ester heavy products and generate an extremely viscous residue, which limits the performance of this cracking and causes problems in the storage and transfer of the residues.

To overcome the problem related to the viscosity, it is also known to add a solvent to the AA heavy products cracking residue.

Document EP 3255030 teaches the addition of higher alcohols during the cleavage of the residue, the maleic anhydride present in the residue being converted into maleic acid esters which are less sensitive to polymerization.

Document U.S. Pat. No. 6,414,183 teaches the dilution of the discharged residue with solvents such as acetic acid, water and methanol.

WO 2021/224044 describes a process for breaking down Michael adducts of acrylic acid, by dilution in a solvent 1 having a boiling point at 1013 hPa of at least 170° C. and a solubility in water at 25° C. of at least 20 g per 100 g of water, said solvent being chosen from alcohols such as ethylene glycol, propylene glycol, diethylene glycol, triethylene glycol and 2-ethoxyethanol, carboxamides such as N,N-dimethylacetamide, N-methylacetamide and N,N-dimethylformamide, sulfoxides such as dimethyl sulfoxide, and sulfones such as sulfolane.

However, this solution has several disadvantages, such as the generation of waste to be burned, if this is not an internal stream, or the provision of additional equipment for performing the mixing. Moreover, most of these solvents generate nitrogen-containing or sulfur-containing derivatives upon burning.

Consequently, there is a need to improve the regeneration efficiency, by thermal cracking, of heavy compounds originating solely from AA units.

### SUMMARY OF THE INVENTION

The invention relates to a process for regenerating a mixture of heavy by-products from an acrylic acid production unit (AAHP), said process comprising the following steps:
i. introducing said heavy by-products with water into a hydrolyzer and subjecting them to continuous hydrolysis, the water:AAHP ratio ranging from 0.1 to 1.3 (limits included) for a period of from 1 to 10 h, preferably from 1 to 5 h, resulting in a mixture of hydrolyzed products being obtained,
ii. continuously injecting said mixture of hydrolyzed products into a reactor and subjecting it to thermal cracking, producing a gaseous overhead stream containing acrylic acid and water and a bottom stream (residue) concentrated in heavy products,
iii. recovering a lighter fraction rich in AA and water that can be recycled to various points in the process,
iv. recovering said residue for the purpose of a removal treatment.

According to various implementations, said process comprises the following characteristics, if appropriate combined.

According to one embodiment, the pressure in the hydrolyzer varies between 0.1 and 2 MPa, preferably between 0.5 and 1.5 MPa.

According to one embodiment, the temperature in the hydrolyzer varies between 80° C. and 200° C., preferably between 150° C. and 200° C.

According to one embodiment, the cracking temperature is between 140° C. and 260° C., preferably between 160° C. and 210° C.

According to one embodiment, the residence time of the reaction mixture in the cracking reactor is between 0.5 h and 10 h, preferably between 4 h and 10 h.

According to one embodiment, the thermal cracking reaction takes place at atmospheric pressure or under slight pressure (maximum 0.2 MPa).

According to one embodiment, the bottom stream from the reactor (residue) obtained on conclusion of the thermal cracking operation has a dynamic viscosity of less than 1 Pa·s, measured at a temperature of 100° C. for example using a Brookfield “CAP 1000+” viscometer of cone/plate type.

According to one embodiment, said gaseous overhead stream containing acrylic acid and water is injected into a condenser.

According to one embodiment, the thermal cracking reaction takes place in the absence of catalyst.

The present invention makes it possible to overcome the disadvantages of the prior art. It makes it possible to recover the maximum amount of AA per cracking operation, while managing the viscosity of the residue formed without being dependent on another production unit. This is accomplished by combining a step of hydrolyzing the heavy by-products from an acrylic acid production unit with a step of thermal cracking of the hydrolyzed products.

The main advantages of the process according to the invention are:
a simple process that is inexpensive in terms of investment since in practice it requires only one additional piece of equipment (the hydrolyzer) compared to the cracking step alone;
a process which makes it possible to reduce the discharges by decreasing the amount of cracking residue;
a lower cracking temperature compared to cracking without a hydrolysis step, therefore enabling energy savings;
a less viscous residue compared to cracking without a hydrolysis step;
the cracking step does not depend on other units (ester units);
the hydrolysis also makes it possible to avoid a special treatment of the residues in order to ensure the discharging thereof, such as the addition of solvent for example in order to fluidify them;
an increase in recovery efficiency of noble products from the heavy Michael derivative products present in the streams of heavy products from AA plants, by pushing the cracking reaction further, the viscosity limit of the residue being reached later compared to a solution without hydrolysis.

### FIGURES

FIG. 1 diagrammatically represents an embodiment of an installation according to the invention.

### DESCRIPTION OF EMBODIMENTS OF THE INVENTION

The invention is now described in greater detail and in a nonlimiting manner in the description that follows.

The term “heavy by-products originating from a unit for the production of acrylic acid” comprises:
derivatives of the addition of acrylic acid to the double bond of another acrylic acid molecule: 3-acryloyloxypropionic acid, also called “acrylic acid dimer” or “AA dimer”;
derivatives of the addition of acrylic acid to the double bond on an AA dimer molecule, to form the “AA trimer”, and other oligomers formed by successive additions of acrylic acid to the double bonds of the preceding AA oligomers;
derivatives of the addition of carboxylic acid formed as by-products of acrylic acid (for example acetic acid) or of water to the double bond of the AA or of the abovementioned oligomers.

The term “hydrolyzer” refers to a reactor in which the hydrolysis reaction of the mixture of water and AA heavy products can be carried out. This reactor can be heated and maintain a pressure. The latter may be a reactor of the conventional stirred type, a plug-flow reactor with or without internal pre-mixer or may be in a heat exchanger.

The invention is based on a thermal cracking process, coupled with a hydrolysis operation carried out beforehand on the heavy by-products from an AA production unit.

The acrylic monomers involved in the Michael addition derivatives can be regenerated by hydrolyzing the oligomers prior to the heat treatment step. This hydrolysis reaction forms hydroxypropionic acid (HPA), which can be thermally cracked to give acrylic acid. Hydrolysis makes it possible to reduce the oligomer chains, making the residue less viscous.

Hydrolysis in continuous mode is carried out under a pressure ranging from 0.1 to 2 MPa.

The regeneration efficiency (expressed as cracking efficiency) depends essentially on:
a/parameters for the hydrolysis: temperature and pressure, hydrolysis residence time and water/AA heavy products ratio, and
b/parameters for the cracking: temperature and residence time of the heat treatment.

The increase in these last two parameters (b/) tends to improve the regeneration efficiency, but this takes place at the expense of an increase in the viscosity of the cracking residue.

Cracking performance is characterized by two values:
the URR or useful recovery rate: this is the amount of acrylic acid recovered after cracking relative to the amount of AA heavy products which feed the cracker:

$$URR = mass of AA recovered / AA heavy products in cracker feed$$

the cracking rate or efficiency: this is the amount of acrylic acid recovered after cracking relative to the sum of the upgradable compounds in the cracker feed (acrylic acid (AA), acrylic acid dimers (AA2) and hydroxypropionic acid (HPA)).

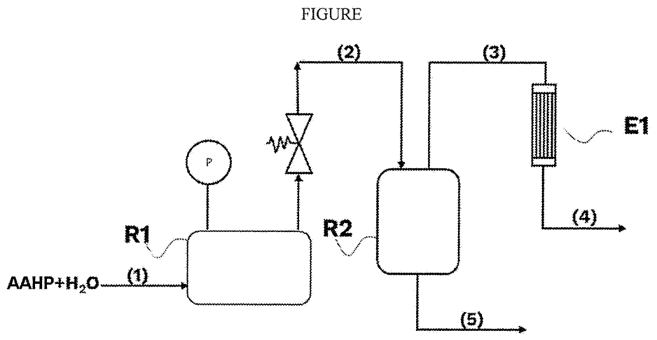

According to the embodiment of the process shown in FIG. 1, the stream containing said heavy by-products from the acrylic acid production plant (AAHP) and water are introduced together or separately in continuous mode into the reactor R1. The AAHP stream is rich in heavy Michael addition derivative compounds generated during the acrylic acid synthesis and purification steps, and also contains other heavy compounds accumulated during the synthesis and purification processes, in particular polymerization inhibitors.

The mixture (1) containing the heavy acrylic acid compounds and water is heated to the temperature required to hydrolyze the Michael addition derivatives to give lighter compounds. The stream (2) leaving the hydrolyzer RI is then introduced continuously into a second reactor R2, where it is heated to the temperature required to crack the Michael addition derivatives to give lighter compounds which are extracted in the form of a gas mixture (3) at the top of the reactor.

This vapor stream rich in acrylic acid and containing some heavy compounds including inhibitors at a low concentration, is advantageously recycled to the acrylic acid production process, either directly in vapor form, or after total condensation in a condenser E1 as stream (4).

According to one embodiment, at least one polymerization inhibitor is introduced into the condenser E1. These inhibitors are chosen from polymerization inhibitors known to a person skilled in the art: phenol derivatives, such as hydroquinone and its derivatives, for instance hydroquinone methyl ether, 2,6-di(tert-butyl)-4-methylphenol (BHT) and 2,4-dimethyl-6-(tert-butyl) phenol (Topanol A), phenothiazine and its derivatives, manganese salts, such as manganese acetate, salts of thiocarbamic or dithiocarbamic acid, such as metal thiocarbamates and dithiocarbamates, for instance copper di(n-butyl) dithiocarbamate, N-oxyl compounds, such as 4-hydroxy-2,2,6,6-tetramethylpiperidineoxyl (4-OH-TEMPO), compounds having nitroso groups, for instance N-nitrosophenylhydroxylamine and its ammonium salts, amine compounds, such as para-phenylenediamine derivatives, or a mixture of these inhibitors.

The residue stream recovered at the reactor bottom (5) is cooled and then removed in the form of a liquid of moderate viscosity, so as to be able to be transported without difficulty by pump, for example as far as a storage tank or an incineration unit.

### EXAMPLES

The following examples illustrate the invention without limiting it.

### Example 1: Continuous Hydrolysis and Cracking (According to the Invention)

The assembly consists of a plug-flow reactor with a total volume of 16.3 cm³, placed in an oil bath heated to 150° C. The hydrolysis is carried out under pressure at 0.4 MPa for a residence time of one hour. The hydrolyzed stream is continuously introduced into a glass reactor with a total volume of 500 cm³, heated by recirculating oil through its double wall, equipped with a side outlet for the outlet of the residue, ensuring a constant usable volume of liquid contained in the reactor of 156.6 cm³.

The reactor is equipped with a stirrer, a temperature probe immersed in the liquid phase, a vertical pipe in the upper part, for the extraction of the vapors, and a condenser. The liquid (distillate) is collected in a receiving flask and analyzed.

Duration of the test: 55 h

Hydrolysis: T=150° C., residence time: 1 h, P=0.4 MPa, water/AA heavy products ratio=0.5

Cracking: T=173° C., atmospheric pressure, residence time: 10 h, water/AA heavy products ratio=0.5

$$URR = 68.95 \%$$

$$Overall cracking rate = 94.85 \%$$

$$Viscosity = 0.785 Pa \cdot s$$

### Example 2 (Comparative): Cracking without Prior Hydrolysis

In this case, the stream of AA heavy products is directly introduced continuously into a glass reactor with a total volume of 500 cm³, heated by recirculating oil through its double wall, equipped with a side outlet for the outlet of the residue, ensuring a constant usable volume of liquid contained in the reactor of 156.6 cm³.

The reactor is equipped with a stirrer, a temperature probe immersed in the liquid phase, a vertical pipe in the upper part, for the extraction of the vapors, and a condenser. The liquid (distillate) is collected in a receiving flask and analyzed.

Cracking: T=183° C., atmospheric pressure, residence time: 10 h

$$URR = 29.2 \%$$

$$Overall cracking rate = 45.1 \%$$

After 24 h, the viscosity is 1.034 Pa·s.

The test was stopped due to clogging of the outlet of the residue after 32 h.

The results obtained are given in table 1 below.

TABLE 1

| | According | |
|---|---|---|
| | to the | |
| | invention | Comparative |
| Test No. | 1 | 2 |
| Hydrolysis - temperature | 150° C. | — |
| Hydrolysis residence time | 1 h | — |
| Water/AAHP ratio | 0.5 | — |
| Cracking temperature | 173° C. | 183° C. |
| Cracking residence time | 10 h | 10 h |
| Useful recovery rate | 68.95% | 29.2% |
| Overall cracking rate | 94.85% | 45.1% |
| Viscosity of the residue | 0.785 | 1.034 |
| in Pa · s @ 100° C. | | |

These comparative examples show that the hydrolysis step enables a significantly improved performance at a lower temperature and the residue generated is less viscous. The addition of water makes it possible to drive the cracking without being confronted with clogging problems.

The hydrolysis step makes it possible to generate a stream, the cracking of which is efficient (high URR and low viscosity) despite a relatively long residence time.

1. A process for regenerating heavy by-products from an acrylic acid production unit (AAHP), said process comprising the following steps:
i. introducing said heavy by-products with water into a hydrolyzer and subjecting said heavy by-products and water to continuous hydrolysis, the water:AAHP ratio ranging from 0.1 to 1.3 for a period of from 1 to 10 h to obtain a mixture of hydrolyzed products,
ii. continuously injecting said mixture of hydrolyzed products into a cracking reactor and subjecting the mixture to thermal cracking, producing a gaseous overhead stream containing acrylic acid (AA) and water and a bottom stream comprising a residue concentrated in heavy products,
iii. recovering a lighter fraction rich in AA and water that can be recycled to various points in the process, and
iv. recovering said residue for the purpose of a removal treatment.

2. The process as claimed in claim 1, wherein step i) is carried out at a pressure of between 0.1 and 2 MPa.

3. The process as claimed in claim 1, wherein a hydrolysis temperature is between 80° C. and 200° C.

4. The process as claimed in claim 1, wherein a cracking temperature is between 140° C. and 260° C.

5. The process as claimed in claim 1, wherein a residence time of the reaction mixture in the cracking reactor is between 0.5 h and 10 h.

6. The process as claimed in claim 1, in which the bottom stream obtained on conclusion of the thermal cracking has a dynamic viscosity, measured at 100° C., of less than 1 Pa·s.

7. The process as claimed in claim 1, further comprising a step of injecting said gaseous overhead stream containing acrylic acid and water into a condenser.

8. The process as claimed in claim 7, wherein at least one polymerization inhibitor is introduced at said condenser.

9. The process as claimed in claim 1, wherein the thermal cracking takes place in the absence of catalyst.

10. The process as claimed in claim 1, wherein step i) comprises subjecting said heavy by-products and water to continuous hydrolysis for a period of from 1 to 5 h.

11. The process as claimed in claim 2, wherein step i) is carried out at a pressure of between 0.5 and 1.5 MPa.

12. The process as claimed in claim 3, wherein the hydrolysis temperature is between 150° C. and 200° C.

13. The process as claimed in claim 4, wherein the cracking temperature is between 160° C. and 210° C.

14. The process as claimed in claim 5, wherein the residence time of the reaction mixture in the cracking reactor is between 4 h and 10 h.
